(19) United States
(12) Patent Application Publication
(10) Pub. No.: US 2009/0227883 A1
(43) Pub. Date: Sep. 10, 2009

(54) AUTOMATED HEART FUNCTION CLASSIFICATION TO STANDARDIZED CLASSES

(76) Inventors: Yunlong Zhang, Mounds View, MN (US); Yi Zhang, Blaine, MN (US); Abhilash Patangay, Inver Grove Heights, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/396,196

(22) Filed: Mar. 2, 2009

Related U.S. Application Data
(60) Provisional application No. 61/033,943, filed on Mar. 5, 2008.

(51) Int. Cl. A61B 5/04; A61B 5/00

(52) U.S. Cl. 600/509; 600/300

(57) ABSTRACT

A system and method automatically classifies a patient's heart function status, such as by using an implantable medical device (IMD) to determine a physiological response to activity, and using that information to perform the classification. For example, a physical activity sensor and a physiological sensor are used to automatically classify patients into heart function status classes, such as NYHA classes or ACC/AHA classes. Changes in a patient's classification can be used to monitor heart function status over time and to monitor therapy responsiveness.

### CROSS-REFERENCE TO RELATED APPLICATION

This application claims the benefit of U.S. Provisional Application No. 61/033,943, filed on Mar. 5, 2008, under 35 U.S.C. §119(e), which is hereby incorporated by reference.

### BACKGROUND

In spite of rapid technological advances, manual New York Heart Association (NYHA) classification by a physician remains the major gauge of heart function assessment in patients with heart disease. In addition to NYHA classification, American College of Cardiology/American Heart Association (ACC/AHA) classification is another method that physicians use for assessing patient heart function status.

### OVERVIEW

This document describes, among other things, a system and method that automatically classifies a patient's heart function status, such as by using an implantable medical device (IMD) to determine a physiological response to activity, and using that information to perform the classification. For example, a physical activity sensor and a physiological sensor are used to automatically classify patients into heart function status classes, such as NYHA classes or ACC/AHA classes. Changes in a patient's classification can be used to monitor heart function status over time and to monitor therapy responsiveness.

Example 1 describes a system. In this example, the system comprises a physical activity sensor, configured to sense an indication of physical activity of a patient; a physiological sensor, configured to sense a physiological response of a patient corresponding to the sensed indication of the physical activity of the patient; a signal processor circuit, configured to receive the indication of physical activity of the patient from the physical activity sensor, and configured to receive the physiological response of the patient from the physiological sensor, and configured to automatically classify the patient into a classification corresponding to a cardiac function status of the patient, the classification selected from a group of standard diagnostic classes describing different cardiac function statuses, the classes recognized by a medical standard-establishing organization; and a patient classification memory storage location, configured to store an indication of the classification of the patient to be provided to a user or process.

In Example 2, the system of Example 1 optionally includes the signal processor circuit configured to repeat the classifying over a period of time, detect a change in the classification during the period of time, and provide an indication of the change in the classification of the patient to a user or process.

In Example 3, the system of one or more of Examples 1-2 optionally includes the signal processor circuit configured to classify the patient into a NYHA class that is automatically selected from a group of NYHA classes using the physiological response to activity.

In Example 4, the system of one or more of Examples 1-3 optionally includes the signal processor circuit configured to classify the patient into an ACC/AHA class that is automatically selected from a group of ACC/AHA classes using the physiological response to activity.

In Example 5, the system of one or more of Examples 1-4 optionally includes the physiological sensor comprising a pH sensor configured to sense pH from the patient.

In Example 6, the system of one or more of Examples 1-5 optionally includes the signal processor circuit configured to use pH to determine an indication of fatigue, and to use the indication of fatigue to automatically classify the patient into a classification corresponding to a cardiac function status of the patient.

In Example 7, the system of one or more of Examples 1-6 optionally includes the physiological sensor comprising a heart rate sensor configured to sense a heart rate of the patient, wherein the signal processor circuit is coupled to the heart rate sensor to receive and use information about the sensed heart rate to automatically classify the patient into a classification corresponding to the cardiac function status of the patient.

In Example 8, the system of one or more of Examples 1-7 optionally includes the physiological sensor comprising a respiration sensor configured to sense a respiration rate of the patient, wherein the signal processor circuit is coupled to the respiration sensor to receive and use information about the sensed respiration rate to automatically classify the patient into a classification corresponding to the cardiac function status of the patient.

In Example 9, the system of one or more of Examples 1-8 optionally includes the physiological sensor comprising a periodic breathing sensor configured to sense a periodic breathing of the patient, wherein the signal processor circuit is coupled to the periodic breathing sensor to receive and use information about the sensed periodic breathing to automatically classify the patient into a classification corresponding to the cardiac function status of the patient.

In Example 10, the system of one or more of Examples 1-9 optionally includes the signal processor configured to compute an indication of the physiological response to activity by: detecting a first measurement of a physiological parameter corresponding to relatively lower degree of physical activity of the patient; detecting a second measurement of the physiological parameter at a relatively greater degree of physical activity of the patient than that corresponding to the first measurement; and determining the physiological response to activity using a change in the physiological parameter between the first and second measurements of the physiological parameter.

In Example 11, the system of one or more of Examples 1-10 optionally includes the signal processor configured to automatically classify the patient into a classification corresponding to a cardiac function status of a patient by processing the measurement of the physiological response to activity using at least one of: patient medication information, patient co-morbidity information, or physician-provided input.

Example 12 describes a method. In this example, the method comprises using a medical device, detecting an indication of physical activity of a patient; using the medical device, detecting a measurement of a physiological response of the patient corresponding to the measurement of physical activity of the patient; using the measurement of the physiological response, automatically classifying the patient into a classification corresponding to a cardiac function status of a patient, the classification selected from a group of standard diagnostic classes describing different cardiac function statuses, the group of classes recognized by a medical standard-establishing organization; and providing an indication of the classification of the patient to a user or process.

In Example 13, the method of Example 12 optionally comprises repeating the classifying over a period of time; detecting a change in the classification during the period of time; and providing an indication of the change in the classification of the patient to a user or process.

In Example 14, the method of one or more of Examples 12-13 optionally comprises classifying the patient into a classification corresponding to cardiac function status of the patient by classifying the patient into a NYHA class that is automatically selected from a group of NYHA classes using the measurement of the physiological response to activity.

In Example 15, the method of one or more of Examples 12-14 optionally comprises classifying the patient into a classification corresponding to cardiac function status of the patient by classifying the patient into an ACC/AHA class that is automatically selected from a group of ACC/AHA classes using the measurement of the physiological response to activity.

In Example 16, the method of one or more of Examples 12-15 optionally comprises detecting the measurement of the physiological response corresponding to the measurement of physical activity by measuring pH.

In Example 17, the method of one or more of Examples 12-16 optionally comprises using measured pH for generating an indication of fatigue, and using the generated indication of fatigue for automatically classifying the patient into the classification corresponding to the cardiac function status of the patient.

In Example 18, the method of one or more of Examples 12-17 optionally comprises detecting the measurement of the physiological response corresponding to the measurement of physical activity by measuring heart rate, wherein classifying the patient into the classification corresponding to a cardiac function status of the patient includes using the measured heart rate.

In Example 19, the method of one or more of Examples 12-18 optionally comprises detecting the measurement of the physiological response corresponding to the measurement of physical activity by measuring respiration rate, wherein classifying the patient into the classification corresponding to a cardiac function status of the patient includes using the measured respiration rate.

In Example 20, the method of one or more of Examples 12-19 optionally comprises detecting the measurement of the physiological response corresponding to the measurement of physical activity by measuring periodic breathing, wherein classifying the patient into the classification corresponding to a cardiac function status of the patient includes using the measured periodic breathing.

In Example 21, the method of one or more of Examples 12-20 optionally comprises detecting the measurement of the physiological response corresponding to the measurement of physical activity by: detecting a first measurement of a physiological parameter corresponding to relatively lower degree of physical activity of the patient; detecting a second measurement of the physiological parameter at a relatively greater degree of physical activity of the patient than that corresponding to the first measurement; and determining the physiological response to activity using a change in the physiological parameter between the first and second measurements of the physiological parameter.

In Example 22, the method of one or more of Examples 12-21 optionally comprises determining a measurement of the physiological response to activity by determining at least one degree of physical activity of the patient using at least one of: a six-minute walk, a maximum exercise intensity level, or a maximum exercise duration.

In Example 23, the method of one or more of Examples 12-22 optionally comprises automatically classifying the patient into a classification corresponding to a cardiac function status of a patient by using the measurement of the physiological response to activity, including processing the measurement of the physiological response using at least one of: patient medication information, patient co-morbidity information, or physician-provided input.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals can describe substantially similar components throughout the several views. Like numerals having different letter suffixes can represent different instances of substantially similar components. The drawings illustrate generally, by way of example, but not by way of limitation, various embodiments discussed in the present document.

FIG. 1 is schematic diagram illustrating generally an example of a cardiac function management system, such as for use with a human or animal subject.

FIG. 2 is a flow chart illustrating generally an example of a technique for automatically classifying a patient into a cardiac function status class.

FIG. 3 is a diagram illustrating generally an example of a system for automatically classifying a patient into a cardiac function status class.

FIG. 4 is a diagram illustrating generally examples of inputs used in a system for classifying a patient into a cardiac function status class.

FIG. 5 is a diagram illustrating generally an example of a system for computing an indication of a patient's physiological response to physical activity.

### DETAILED DESCRIPTION

This document describes, among other things, automatic classification of a patient into a heart function status class, such as by using an implantable medical device that measures a physiological response to physical activity. Such information can be used to classify the patient into a medically recognized standardized heart function class.

Table 1 illustrates NYHA classification, a standardized medically-recognized schema that is typically used by doctors for classifying heart status manually, rather than automatically using physiological response to activity information obtained from an implantable medical device, as described below. Advancement to a higher-numbered NYHA class is generally accompanied by increased heart failure mortality of the subpopulation represented by that class. NYHA Class II patients generally exhibit a heart failure mortality rate of 5-10%, Class III patients generally exhibit a heart failure mortality rate of 10-15%, and Class IV patients generally exhibit a heart failure mortality rate of 30-40%.

TABLE 1
NYHA classification

| Class | Patient Symptoms |
|---|---|
| Class I | No limitations of physical activity. Ordinary physical activity |
| | does not cause undue fatigue, palpitation, or dyspnea. |
| Class II | Slight limitation of physical activity. Comfortable at rest, but |
| | ordinary physical activity results in fatigue, palpitation, or |
| | dyspnea. |
| Class III | Marked limitation of physical activity. Comfortable at rest, |
| | but less than ordinary activity causes fatigue, palpitation, |
| | or dyspnea. |
| Class IV | Unable to carry out any physical activity without discomfort. |
| | Symptoms of cardiac insufficiency at rest. If any physical |
| | activity is undertaken, discomfort is increased. |

Table 2 illustrates ACC/AHA classification based on a patient's symptoms and the physical condition of the patient's heart. The ACC/AHA classification schema is a standardized medically-recognized schema that is typically used by doctors for classifying heart status manually, rather than automatically using physiological response to activity information obtained from an implantable medical device, as described below. At the present time, ACC/AHA stages may be thought of as being less dynamic the NYHA classes. For example, once a patient is classified as ACC/AHA stage B, the patient generally cannot improve to stage A, even if that patient's NYHA classification improves. In the future, however, technology may allow for earlier detection and reversal of heart failure signs, which would permit patients to improve from one ACC/AHA stage to the next. In either case, long-term monitoring of ACC/AHA stages may be useful.

TABLE 2
ACC/AHA Heart Failure (HF) classification schema

| Stage | Description | Examples |
|---|---|---|
| A | Patients at high risk of developing | Systemic hypertension; coronary |
| | HF because of the presence of | artery disease; diabetes mellitus; |
| | conditions that are strongly | history of cardiotoxic drug therapy |
| | associated with the development | or alcohol abuse; personal history of |
| | of HF. Such patients have no | rheumatic fever; family history of |
| | identified structural or functional | cardiomyopathy. |
| | abnormalities of the pericardium, | |
| | myocardium, or cardiac valves | |
| | and have never shown signs or | |
| | symptoms of HF. | |
| B | Patients who have developed | Left ventricular hypertrophy or |
| | structural heart disease that is | fibrosis; left ventricular dilation or |
| | strongly associated with the | hypocontractility; asymptomatic |
| | development of HF but who have | valvular heart disease; previous myocardial |
| | never shown signs or symptoms of | infarction. |
| | HF. | |
| C | Patients who have current or prior | Dyspnea or fatigue due to left |
| | symptoms of HF associated with | ventricular systolic dysfunction; |
| | underlying structural heart | asymptomatic patients who are |
| | disease. | undergoing treatment for prior |
| | | symptoms of HF. |
| D | Patients with advanced structural | Patients who are frequently |
| | heart disease and marked | hospitalized for HF or cannot be |
| | symptoms of HF at rest despite | safely discharged from the hospital; |
| | maximal medical therapy and who | patients in the hospital awaiting |
| | require specialized interventions. | heart transplantation; patients at |
| | | home receiving continuous |
| | | intravenous support for symptom |
| | | relief or being supported with |
| | | mechanical circulatory assist device; |
| | | patients in a hospice setting for the |
| | | management of HF. |

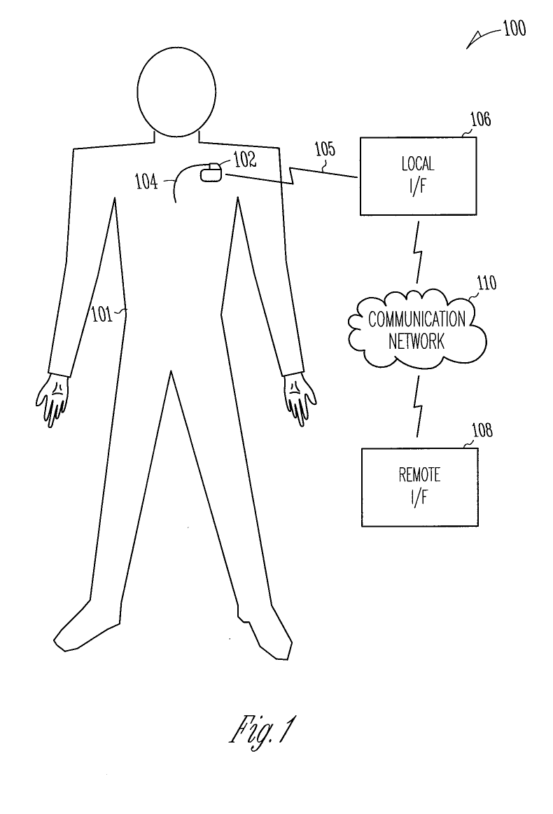

FIG. 1 is schematic diagram illustrating generally an example of a cardiac function management system 100, such as for use with a human or animal subject 101. In this example, the system 100 includes an implantable cardiac function management device 102, which can include or be coupled to one or more intravascular or other leads 104. The cardiac function management device 102 can include a communication circuit, such as for establishing a bidirectional wireless communication link 105 with an external local interface 106. In certain examples, the external local interface can further bidirectionally communicate with an external remote interface 108, wirelessly or otherwise, such as via a shared communication or computer network 110. An example of using such a communication network 110 can include using the Boston Scientific Corp. (Cardiac Pacemakers, Inc.) LATITUDE® Patient Monitoring System, which can provide remote patient monitoring, such as by automatically collecting information from a patient's implanted medical device and communicating the information to a secure website accessible by the patient's healthcare providers.

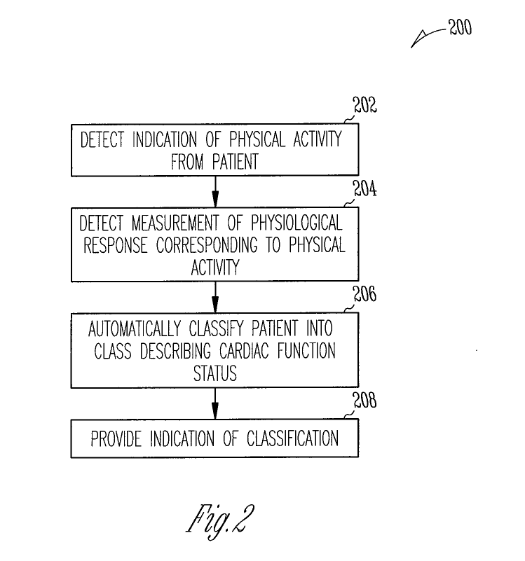

FIG. 2 is a flow chart illustrating generally an example of a technique 200 for automatically classifying a patient into a cardiac function status class based on the patient's physiological response to physical activity. Some examples of measuring a patient's physiological response to physical activity are described in Beck et al., U.S. Patent Application Serial No. US 2007/0021678 entitled “Methods and Apparatus for Monitoring Physiological Responses to Steady State Activity” (Attorney Docket No. 279.916US1), assigned to Cardiac Pacemakers, Inc., and filed on Jul. 19, 2005, which is incorporated herein by reference in its entirety, including its description of measuring a patient's physiological response to physical activity. At 202, an indication of physical activity is detected from the patient. The indication of physical activity can be generated, for example, by using one or more implantable movement or exertion sensors, such as an accelerometer. At 204, a measurement of physiological response corresponding to the physical activity is detected from the patient. The measurement of a physiological response to the physical activity can be generated by one or more physiological sensors, such as an implantable pH sensor, a heart rate sensor, a respiration sensor, or a periodic breathing sensor, for example. At 206, the patient is automatically classified into a class describing cardiac function status. The classification can be based on the indication of physical activity 202 and the measurement of physiological response 204. In certain examples, the classification can be based on baseline measurements of a patient's physiological response to physical activity. Baseline measurements are measurements of a physiological response to a physical activity at a particular point in time. Baseline measurements can later be compared to physiological responses measured at other times in order to detect relative changes. A six-minute walk test, for example, can be used to establish baseline measurements of a patient's pH, heart rate, and respiration rate. These baseline measurements can then be used to set one or more parameters used in automatically classifying a particular patient's heart status. For example, when a patient is initially classified into a cardiac function class using the baseline measurements, the parameters for later classifications can then be determined using the patient's initial classification. Other information such as co-morbidities or medications can also be used to determine the parameters used for later classifications. Cardiac function classes can include medically-recognized standard diagnostic classes, such as NYHA classes or ACC/AHA classes. At 208, an indication of the patient's automatic heart status classification is provided to a user or process, such as through a communication network 110. The classification indication can be stored in a memory storage location, or displayed to the user, in certain examples. In the example of FIG. 2, the detecting the indication of physical activity 202 and the detecting the measurement of physiological response corresponding to physical activity 204 can be performed internally within a subject's body using an implantable cardiac function management device. The automatic heart status classification of the patient 206 and the generation of an indication of classification 208 can be performed internally within the implantable device, or externally, such as within a local or remote user interface device.

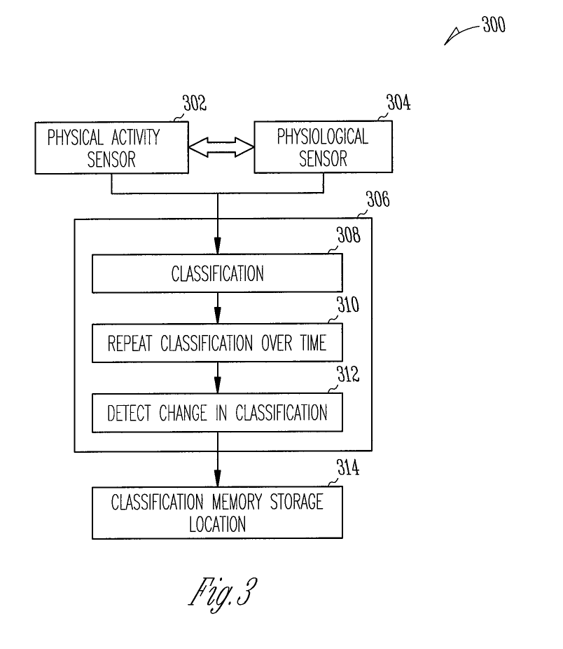

FIG. 3 is a diagram illustrating generally an example of a system 300 for automatically classifying a patient into a cardiac function status class, such as based on the patient's physiological response to physical activity. In this example, a physical activity sensor 302 is configured to sense an indication of physical activity of a patient. The indication of physical activity can be sensed, for example, using an accelerometer or an exertion or movement sensor. A physiological sensor 304 can be configured to sense a physiological response of the patient corresponding to the sensed indication of the patient's physical activity. The measurement of physiological response can be generated by one or more physiological sensors, such as an implantable pH sensor, a heart rate sensor, a respiration sensor, or a periodic breathing sensor. Information from the physical activity sensor and from the physiological sensor is communicated to the classification circuit 308 within the signal processor circuit 306. The classification circuit 308 automatically classifies the patient into a class corresponding to the cardiac function status of the patient. For example, the classification circuit 308 can classify the patient into one or more of a NYHA class or an ACC/AHA class. In addition to the classification circuit 308, the signal processor circuit 306 can be configured to repeat the classification process over an acute or chronic period of time 310 such as to detect a change in heart status classification 312. Changes in heart status classification automatically detected using the signal processor circuit 306 can be communicated to a classification memory storage location 314 configured to store such heart status classifications of the patient, such as for determining an indication of a change in the heart status classification over an acute or chronic period of time. Such changes in heart status classification over time can be used to monitor heart function status or to monitor therapy effectiveness or responsiveness. Detection of frequent changes in heart status classification or of worsening heart status classification can be communicated to a patient or caregiver through the generation of local or remote alerts or alarms. Automatic therapy changes can be made in response to a detected worsening, improvement, or other change in heart function status classification. In the example of FIG. 3, the physical activity sensor 302 and the physiological sensor 304 can be implantable, for example, included within or implantably coupled to an implantable cardiac function management device. The signal processor circuit 306 and the classification memory storage location 314 can be implantably located, such as within the implantable cardiac management device, or externally located.

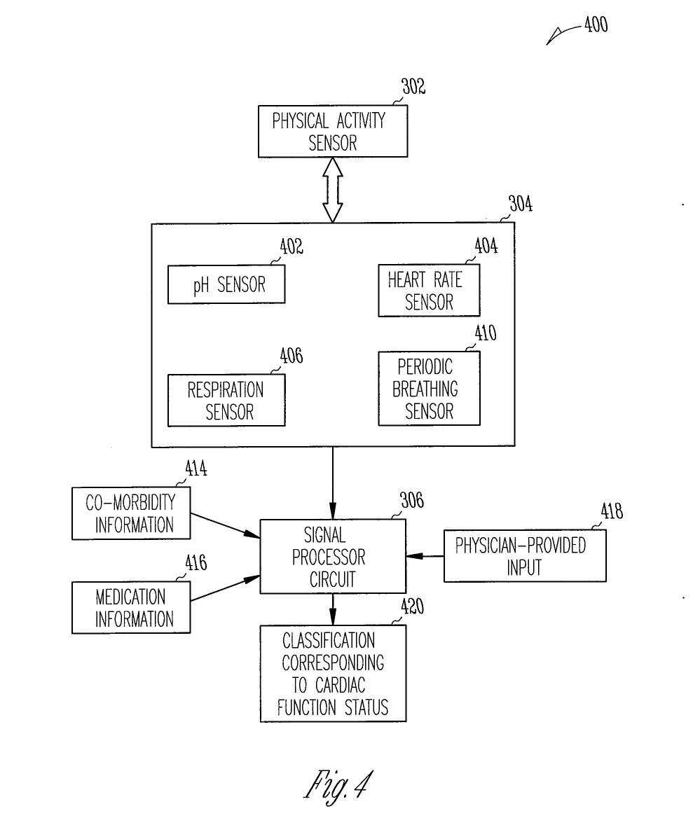

FIG. 4 is a diagram illustrating generally an example of a system 400 in which a patient can be classified, such as according to heart status using information from the physical activity sensor 302 and the physiological sensor 304, although additional inputs can also be used. In this example, the physiological sensor 304 can include one or more different sensors of respective physiological parameters, such as a pH sensor 402, a heart rate sensor 404, a respiration sensor 406, or a periodic breathing sensor 410. The pH sensor 402 can be configured to detect pH or other measure of acidity or alkalinity in the blood stream or in muscle tissue, such as pectoral muscle tissue or at skeletal muscle tissue of the lower limb. The pH sensor 402 can be configured to detect pH using one or more of pH electrodes or optical pH sensors, for example. A decrease in pH generally accompanies muscle fatigue, which can signal worsening heart function status, particularly when the muscle fatigue generally increases during a period of time in which the patient's physical activity level has not shown any increase. The heart rate sensor 404 can detect increased heart rate and arrhythmias, both of which can be indications of worsening cardiac function status, particularly when the patient's physical activity level has not increased. The respiration sensor 406 can detect increased respiration rate, another indication of worsening heart function status, particularly when the patient's physical activity level has not increased. The periodic breathing sensor 410 can be used to detect one or more signs of dyspnea, such as a periodically decreased tidal volume. An increasing degree of dyspnea can provide another indication of worsening cardiac function status.

Information about one or more of the physiological parameters measured by one or more of the various sensors can be communicated from the physiological sensor 304 to the signal processor circuit 306. Using the information about the patient's physiological response to physical activity, the signal processor 306 can be configured to automatically classify the patient into a class corresponding to cardiac function status 420. In addition to physiological response to physical activity data from the physiological sensor 306, the signal processor circuit 306 can use patient co-morbidity information 414, patient medication information 416, and physician-provided input 418 to automatically classify the patient into a class corresponding to cardiac function status 420. For example, from the outset, a patient who has chronic obstructive pulmonary disease (COPD), in addition to a heart failure condition, may exhibit, in response to an increase in physical activity, a bigger increase in respiration or heart rate, or a bigger decrease in pH relative to a patient having a heart failure condition without the accompanying COPD co-morbidity. These COPD-related effects can be taken into account by the signal processor circuit 306 in classifying the patient according to heart function status. Furthermore, certain medications can affect a patient's physiologic response to physical activity. For example, patients taking beta blockers generally exhibit a lesser increase in heart rate in response to physical activity compared to patients who are not on beta blockers. Thus, for a patient taking beta-blockers, the signal processor circuit 306 can be programmed to allow for a lower heart rate threshold for placing a patient into a “more compromised” heart status class when classifying the patient according to cardiac function status. In certain examples, a physician can independently classify a patient into a heart status class based on one or more of the patient's symptoms and response to a six-minute walk test, without using the patient's implanted automatic heart function status classification device. In certain examples, the physician's independent classification can be used as an input signal for the signal processor circuit 306, and the automatic classification can be compared to the physician's classification. The physician's independent classification or the results of a patient's six-minute walk test can be used to adjust the automatic classification system for a particular patient, such as to calibrate the automatic classification system or to make the automatic classification system adaptive via a machine learning process, for example. Physician calibration can be performed recurrently or periodically.

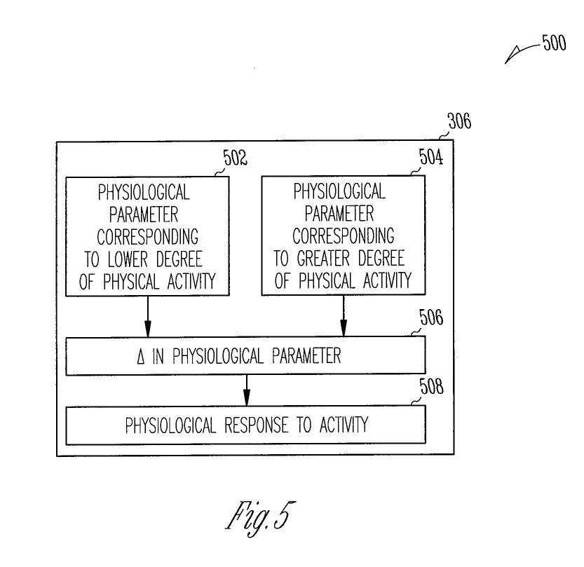

FIG. 5 is a diagram illustrating generally an example of a system 500 in which the signal processor circuit 306 is configured to compute an indication of the physiological response to activity 508. At 502, the signal processor circuit 306 detects a physiological parameter corresponding to a lower degree of physical activity. At 504, the signal processor circuit 306 detects the physiological parameter corresponding to a higher degree of physical activity. At 506, the physiological parameter corresponding to the lower degree of physical activity 502 is compared to the physiological parameter corresponding to the higher degree of physical activity 504, and the change in the physiological parameter is determined. At 508, the physiological response to activity is determined using the change in the physiological parameter 506 between the lower and higher physical activity measurements. In certain examples, the physiological response is measured at steady-state values of physical activity, for example, such as described in the above-incorporated Beck et al. patent application. The corresponding physiological response to activity can then be used to classify the patient into a heart status class, such as described above.

Table 3 is an example of an automatic machine-implemented NYHA classification based on patient respiration rate, such as described above. In certain examples, a patient can be automatically classified into one of the four NYHA classes depending on that patient's measured respiration rate during various levels of physical activity. Both the respiration rate and the physical activity level can be measured using an implantable medical device, such as described below. The automatic heart status classification can then be performed using the implantable or an external device, such as described above. The numbers provided in this table are non-limiting illustrative examples.

TABLE 3
Automatic classification into NYHA classes using respiration as the
physiological response to physical activity.

| | Physical Activity and Symptom (Dyspnea) Relationship | | |
|---|---|---|---|
| | Ordinary physical | Less than ordinary | |
| | activity | physical activity | Rest |
| | (accelerometer | (accelerometer scale | (accelerometer |
| Class | scale >80 mg) | 15-80 mg) | scale <15 mg) |
| Class I | RR ≦20 bpm | RR ≦20 bpm | RR ≦20 bpm |
| Class II | RR 21-25 bpm | RR ≦20 bpm | RR ≦20 bpm |
| Class III | RR 26-30 bpm | RR 21-25 bpm | RR ≦20 bpm |
| Class IV | RR >30 bpm | RR >25 bpm | RR >20 bpm |

Table 4 is an example of an automatic machine-implementable NYHA classification based on patient heart rate. In certain examples, a patient can be automatically classified into one of the four NYHA classes depending on that patient's measured heart rate during various levels of physical activity. Both the heart rate and the physical activity level can be measured using an implantable medical device, such as described above. The automatic heart status classification can then be performed using the implantable or an external device, such as described above. The numbers provided in this table are non-limiting illustrative examples.

TABLE 4
Automated classification to NYHA classes using heart rate as the
physiological response to physical activity.

| | Physical Activity and Symptom (Palpitation) Relationship | | |
|---|---|---|---|
| | Ordinary physical | Less than ordinary | |
| | activity | physical activity | Rest |
| | (accelerometer | (accelerometer scale | (accelerometer |
| Class | scale >80 mg) | 15-80 mg) | scale <15 mg) |
| Class I | HR ≦90 bpm | HR ≦90 bpm | HR ≦90 bpm |
| Class II | HR 91-100 bpm | HR ≦90 bpm | HR ≦90 bpm |
| Class III | HR 101-120 bpm | HR 91-100 bpm | HR ≦90 bpm |
| Class IV | HR >120 bpm | HR >100 bpm | HR >100 bpm |

### Additional Notes

In this document, certain examples have been described with respect to using a “respiration rate measurement,” for illustrative clarity. However, such examples can also be performed using a “respiration interval measurement” rather than a “respiration rate measurement,” without departing from the scope of the described systems and methods.

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B.” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAM's), read only memories (ROM's), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system comprising:
a physical activity sensor, configured to sense an indication of physical activity of a patient;
a physiological sensor, configured to sense a physiological response of a patient corresponding to the sensed indication of the physical activity of the patient;
a signal processor circuit, configured to receive the indication of physical activity of the patient from the physical activity sensor, and configured to receive the physiological response of the patient from the physiological sensor, and configured to automatically classify the patient into a classification corresponding to a cardiac function status of the patient, the classification selected from a group of standard diagnostic classes describing different cardiac function statuses, the classes recognized by a medical standard-establishing organization; and
a patient classification memory storage location, configured to store an indication of the classification of the patient to be provided to a user or process.

2. The system of claim 1, wherein the signal processor circuit is configured to repeat the classifying over a period of time, and wherein the signal processor circuit is configured to detect a change in the classification during the period of time, and wherein the system is configured to provide an indication of the change in the classification of the patient to a user or process.

3. The system of claim 1, wherein the signal processor circuit is configured to classify the patient into a NYHA class that is automatically selected from a group of NYHA classes using the physiological response to activity.

4. The system of claim 1, wherein the signal processor circuit is configured to classify the patient into an ACC/AHA class that is automatically selected from a group of ACC/AHA classes using the physiological response to activity.

5. The system of claim 1, wherein the physiological sensor comprises a pH sensor configured to sense a pH from the patient.

6. The system of claim 5, wherein the signal processor circuit is configured to use the pH to determine an indication of fatigue, and to use the indication of fatigue to automatically classify the patient into a classification corresponding to a cardiac function status of the patient.

7. The system of claim 1, wherein the physiological sensor comprises a heart rate sensor configured to sense a heart rate of the patient, and wherein the signal processor circuit is coupled to the heart rate sensor to receive and use information about the sensed heart rate to automatically classify the patient into a classification corresponding to the cardiac function status of the patient.

8. The system of claim 1, wherein the physiological sensor comprises a respiration sensor configured to sense a respiration rate of the patient, and wherein the signal processor circuit is coupled to the respiration sensor to receive and use information about the sensed respiration rate to automatically classify the patient into a classification corresponding to the cardiac function status of the patient.

9. The system of claim 1, wherein the physiological sensor comprises a periodic breathing sensor configured to sense a periodic breathing of the patient, and wherein the signal processor circuit is coupled to the periodic breathing sensor to receive and use information about the sensed periodic breathing to automatically classify the patient into a classification corresponding to the cardiac function status of the patient.

10. The system of claim 1, wherein the signal processor is configured to compute an indication of the physiological response to activity by:
detecting a first measurement of a physiological parameter corresponding to a relatively lower degree of physical activity of the patient;
detecting a second measurement of the physiological parameter at a relatively greater degree of physical activity of the patient than that corresponding to the first measurement; and
determining the physiological response to activity using a change in the physiological parameter between the first and second measurements of the physiological parameter.

11. The system of claim 1, wherein the signal processor is configured to automatically classify the patient into a classification corresponding to a cardiac function status of a patient by processing the measurement of the physiological response to activity using at least one of: patient medication information, patient co-morbidity information, or physician-provided input.

12. A method comprising:
using a medical device, detecting an indication of physical activity of a patient;
using the medical device, detecting a measurement of a physiological response of the patient corresponding to the measurement of physical activity of the patient;
using the measurement of the physiological response, automatically classifying the patient into a classification corresponding to a cardiac function status of a patient, the classification selected from a group of standard diagnostic classes describing different cardiac function statuses, the group of classes recognized by a medical standard-establishing organization; and
providing an indication of the classification of the patient to a user or process.

13. The method of claim 12, comprising:
repeating the classifying over a period of time;
detecting a change in the classification during the period of time; and
providing an indication of the change in the classification of the patient to a user or process.

14. The method of claim 12, wherein classifying the patient into a classification corresponding to the cardiac function status of the patient comprises classifying the patient into a NYHA class that is automatically selected from a group of NYHA classes using the measurement of the physiological response to activity.

15. The method of claim 12, wherein classifying the patient into a classification corresponding to cardiac function status of the patient comprises classifying the patient into an ACC/AHA class that is automatically selected from a group of ACC/AHA classes using the measurement of the physiological response to activity.

16. The method of claim 12, wherein detecting the measurement of the physiological response corresponding to the measurement of physical activity comprises measuring pH.

17. The method of claim 16, comprising:
using the measured pH for generating an indication of fatigue; and
using the generated indication of fatigue for automatically classifying the patient into the classification corresponding to the cardiac function status of the patient.

18. The method of claim 12, wherein detecting the measurement of the physiological response corresponding to the measurement of physical activity comprises measuring heart rate, and wherein classifying the patient into the classification corresponding to a cardiac function status of the patient includes using the measured heart rate.

19. The method of claim 12, wherein detecting the measurement of the physiological response corresponding to the measurement of physical activity comprises measuring respiration rate, and wherein classifying the patient into the classification corresponding to a cardiac function status of the patient includes using the measured respiration rate.

20. The method of claim 12, wherein detecting the measurement of the physiological response corresponding to the measurement of physical activity comprises measuring periodic breathing, and wherein classifying the patient into the classification corresponding to a cardiac function status of the patient includes using the measured periodic breathing.

21. The method of claim 12, wherein detecting the measurement of the physiological response corresponding to the measurement of physical activity comprises:
detecting a first measurement of a physiological parameter corresponding to a relatively lower degree of physical activity of the patient;
detecting a second measurement of the physiological parameter at a relatively greater degree of physical activity of the patient than that corresponding to the first measurement; and
determining the physiological response to activity using a change in the physiological parameter between the first and second measurements of the physiological parameter.

22. The method of claim 21, wherein determining the measurement of the physiological response to activity comprises determining at least one degree of physical activity of the patient using at least one of: a six-minute walk, a maximum exercise intensity level, or a maximum exercise duration.

23. The method of claim 12, wherein automatically classifying the patient into a classification corresponding to a cardiac function status of a patient comprises using the measurement of the physiological response to activity, including processing the measurement of the physiological response using at least one of: patient medication information, patient co-morbidity information, or physician-provided input.
